(19) United States
(12) Patent Application Publication
(10) Pub. No.: US 2024/0374486 A1
(43) Pub. Date: Nov. 14, 2024

(54) COSMETIC MAKE-UP COMPOSITION

(71) Applicant: L V M H RECHERCHE, SAINT-JEAN DE BRAYE (FR)

(72) Inventors: Leïla AZZAZ, SAINT-JEAN DE BRAYE (FR); Naima CHENTOUFI, SAINT-JEAN DE BRAYE (FR); Brigitte NOE, SAINT-JEAN DE BRAYE (FR); Anny MURA, SAINT-JEAN DE BRAYE (FR)

(73) Assignee: L V M H RECHERCHE, SAINT-JEAN DE BRAYE (FR)

(21) Appl. No.: 18/575,604

(22) PCT Filed: Jun. 30, 2022
(86) PCT No.: PCT/FR2022/051315

§ 371 Date: Dec. 29, 2023

(30) Foreign Application Priority Data
Jul. 2, 2021 (FR) FR2107198

(51) Int. Cl. A61K 8/06; A61K 8/81; A61K 8/891; A61K 8/895; A61K 8/92; A61Q 1/02

(52) U.S. Cl. CPC A61K 8/064; A61K 8/8147; A61K 8/8152; A61K 8/891; A61K 8/895; A61K 8/92; A61Q 1/02; A61K 2800/43; A61K 2800/544; A61K 2800/594; A61K 2800/622

(57) ABSTRACT

The present invention relates to a cosmetic make-up composition for keratinous materials, in particular the skin, in the form of a water-in-oil emulsion, comprising, in a physiologically acceptable medium, at least:
a) a vinyl polymer having at least one unit derived from carbosiloxane dendrimer,
b) a silicone acrylate polymer different from compound a),
c) one or more volatile oils,
d) a C8-C22 alkyl dimethicone, and
e) colouring materials, surface-treated by a hydrophobic agent.

### FIELD OF THE INVENTION

The present invention relates to cosmetic make-up compositions for the skin, in particular fluid foundations giving the skin improved colour properties (colour fidelity and stability) without compromising holding (non-transfer).

### PRIOR ART

Make-up compositions for the skin, in particular foundations, have been used for many years to improve the aesthetic appearance of the skin by reducing, in particular, visible and/or tactile irregularities of the skin. Today, consumers are looking for lighter, comfortable galenics, with a natural make-up result, but without compromising the make-up result (covering but natural) and its holding over time (preferably holding for 24 hours). It is known from the prior art to use water-in-oil emulsions comprising silicone oils and hydrophobic-treated pigments for good adhesion on the skin, and film-forming polymers in order to improve the holding of the deposit, but there remains a need to develop novel compositions with improved colour properties (colour fidelity and stability), without compromising the holding (non-transfer)).

The applicant has fittingly demonstrated that the use of a C8-C22 alkyl dimethicone, in particular a cetyl dimethicone, in a water-in-oil emulsion comprising two distinct silicone acrylate polymers, volatile oils and pigments treated by a hydrophobic agent, would enable these expectations to be met.

### DISCLOSURE OF THE INVENTION

A first object of the invention is therefore a cosmetic make-up composition for keratinous materials, in particular the skin, in the form of a water-in-oil emulsion, comprising, in a physiologically acceptable medium, at least:
a) a vinyl polymer having at least one unit derived from carbosiloxane dendrimer,
b) a silicone acrylate polymer distinct from compound a),
c) one or more volatile oils,
d) a C8-C22 alkyl dimethicone, and
e) colouring materials, surface-treated by a hydrophobic agent.

The term “vinyl polymer having at least one unit derived from carbosiloxane dendrimer” shall mean that the vinyl polymer has a molecular side chain containing a carbosiloxane dendrimer structure.

The invention also relates to a cosmetic method for make-up of keratinous materials, in particular the skin, preferably the skin of the face and/or the neck, comprising applying, on said keratinous material, at least one cosmetic composition as defined according to the invention.

### DETAILED DESCRIPTION OF THE INVENTION

The invention therefore relates, in particular, to a cosmetic make-up composition for keratinous materials, in particular the skin, in the form of a water-in-oil emulsion, comprising, in a physiologically acceptable medium, at least:
a) a vinyl polymer having at least one unit derived from carbosiloxane dendrimer,
b) a silicone acrylate polymer distinct from compound a),
c) one or more volatile oils,
d) a C8-C22 alkyl dimethicone, and
e) colouring materials, surface-treated by a hydrophobic agent.

The presence of the C8-C22 alkyl dimethicone, in particular a cetyl dimethicone improves the colour placement at application and the stability of the colour over time.

The term “colour placement” at application according to the invention means the make-up result (colour) obtained after putting on. The final result is generally obtained in between 5 and 30 minutes. A colour is sought after application which is as faithful as possible to the colour at the outlet of the pump (colour in the bulk). Reference is made to the ‘colour fidelity’ at application.

The term “rise in colour” over time according to the invention, means the change in the colour over the course of the day (6 to 8 hours after application). More specifically, once placed (cf. above), the colour may change (to a greater or lesser degree) over the course of the day, depending on the stability of the film (texture) and the interaction with the skin (secretion of sebum, etc.). A colour is sought after 6 to 8 h that is as faithful as possible to the colour after application, in other words a low rise in colour over time. Reference is made to “colour fidelity” or “colour stability” over time.

### Oily Phase

The composition of the invention comprises at least one oily phase.

The term “oily phase” shall mean an oil or a mixture of mutually miscible oils.

In the context of the invention, the term “oil” shall mean a fatty substance that is not soluble in water and that is liquid at 25° C. and atmospheric pressure.

An oily phase according to the invention may comprise fluorinated or non-fluorinated, silicone hydrocarbon oils, and the mixtures thereof. The composition of the invention preferably comprises at least silicone oils and optionally also hydrocarbon oils.

In the context of the invention, “hydrocarbon oil” shall mean an oil containing mainly hydrogen and carbon atoms.

In the context of the invention, ‘silicone oil’ shall mean an oil comprising at least one silicon atom, and in particular an Si—O group.

In the context of the invention, ‘fluorinated oil’ shall mean an oil comprising at least one fluorine atom.

These oils can be volatile or non-volatile, vegetable, mineral or synthetic.

In the context of the invention, the term “volatile oil” shall mean an oil having lost more than 20% of its mass after 15 minutes, more than 40% of its mass after 30 minutes and more than 70% of its mass after 60 minutes, according to the following protocol: 20 mg of the oil to be studied is weighed on a plate of PMMA of 5 cm×5 cm using a micropipette and a precision balance. This material is spread by finger over the entire plate. Said plate is then deposited in a thermostatic ventilated chamber at 25° C. and 50% humidity. The test is carried out three times for each material. The mass loss during drying is measured after 15 minutes, 30 minutes and 60 minutes. The mass loss is expressed according to the following calculation:

$$\% mass loss = \frac{{m}_{t 0} - {m}_{tx}}{{m}_{t 0}} \times 100 \qquad [ Math . 1 ]$$

with mₜₓ corresponding to the mass remaining at the time of measurement (t15 min, t30 min or t60 min) and mₜ₀ corresponding to the initially applied mass.

In the context of the invention, the term “non-volatile oil” shall mean an oil which does not correspond to the above-described definition of a “volatile oil”.

The oils can be present in the composition of the invention at a content ranging from 10 to 20 wt % relative to the total weight of the composition. In the remainder of the description, the indicated oil contents, which relates to the non-volatile oil or volatile oil contents, can also take account of the oil contents present in the raw materials as well as solvents, for example for dispersing compounds such as gelling agents, film-forming polymers, and pigments.

### Volatile Oils

The composition of the invention comprises one or more volatile oils, preferably at a total content ranging from 5 to 60 wt %, more preferably 10 to 35 wt %, preferably 15 to 30 wt %, relative to the total weight of said composition.

According to a particular embodiment, the one or more volatile oils are present in the composition at a total content de 5 to 60 wt %, preferably at a total content of at least 10 wt %, preferably at least 20 wt %, more preferably at least 25 wt %, relative to the total weight of said composition.

The large amount of volatile phase, and in particular of volatile oils makes it possible to have a light composition that is easily applied on the skin; the volatile oils participate in the placing of the film on the skin during application and, when they evaporate, they leave the film adhering to the skin, with a sensation of naked skin, without material effect or masking effect on the skin.

Volatile hydrocarbon oils include, in particular, C₈-C₁₆ branched alkanes, C₈-C₁₆ branched ester, and the mixtures thereof.

Volatile silicone oils include, in particular, linear volatile silicone oils.

The volatile oils which are used in the composition of the invention are preferably volatile silicones or saturated branched-chain hydrocarbons.

The volatile oil can, in particular, be chosen from silicone oils such as dimethicones (polydimethylsiloxanes) the viscosity of which ranges from 0.5 to 6 cSt, alkyl trisiloxanes and cyclomethicones. These include, for example, hexamethyl disiloxane, heptamethyl hexyltrisiloxane, heptamethyloctyl trisiloxane, octamethyl trisiloxane, 1,1,1,3,5,5,5-heptamethyl-3-(trimethylsilyloxy)trisiloxane (alternatively called methyl trimethicone), decamethyl tetrasiloxane, dodecamethyl pentasiloxane, and the mixtures thereof.

According to a particular preferred embodiment, the composition of the invention comprises at least one volatile silicone oil chosen from dimethicones, the viscosity of which ranges from 0.5 to 6 cSt, methyl trimethicone and the mixtures thereof.

The volatile hydrocarbon oil can be isohexyl neopentanoate or a hydrocarbon such as isododecane, isodecane, isohexadecane, n-dodecane (C₁₂) and the n-tetradecane (C₁₄) or the mixtures thereof.

According to a particular embodiment, the composition of the invention comprises at least isododecane.

According to a particular preferred embodiment, the composition of the invention comprises at least two silicone oils volatiles (5-cst dimethicone and methyl trimethicone) and a volatile hydrocarbon oil (isododecane).

According to a particular embodiment, the composition of the invention comprises a non-volatile oil content less than 10%, in particular less than 5%, or even is free from non-volatile oil.

Vinyl polymer with carbosiloxane dendrimer unit (silicone acrylate polymer with dendrimer unit)

The composition of the invention comprises a silicone acrylate polymer with dendrimer unit, in particular a vinyl polymer with carbosiloxane dendrimer unit.

The vinyl polymer possesses, in particular, a backbone and at least one side chain, which comprises a unit derived from carbosiloxane dendrimer having a carbosiloxane dendrimer structure, such as those described in the applications WO03/045337 and EP 963 751 from Dow Chemical.

In the context of the present invention, the term “carbosiloxane dendrimer structure” represents a molecular structure possessing branched groups having high molecular masses, said structure having a high regularity in the radial direction starting from the bond with the backbone. Such carbosiloxane dendrimer structures are described in the form of a strongly branched siloxane-silyl alkylene copolymer in the Japanese patent application filed under number JP H09-171 154 and published under number JP H11-001530.

A vinyl polymer having at least one unit derived from carbosiloxane dendrimer, possesses a molecular side-chain containing a carbosiloxane dendrimer structure, and can originate from the polymerisation of:
(A) 0 to 99.9 parts by weight of a vinyl monomer; and
(B) 100 to 0.1 parts by weight of a carbosiloxane dendrimer containing an organic group that can be polymerised using radicals, represented by the general formula (I):

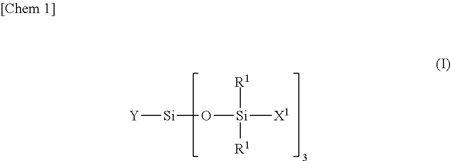

in which:
Y represents an organic group that can be polymerised by radicals,
R1 represents an aryl group having 5 to 10 carbon atoms or an alkyl group possessing 1 to 10 carbon atoms, and
Xi represents a silyl alkyl group which, when i=1, is represented by the formula (II):

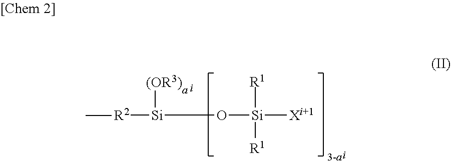

in which:
R¹ is as in formula (I),
R² represents an alkylene group possessing 2 to 10 carbon atoms,
R³ represents an alkyl group possessing 1 to 10 carbon atoms,
Xⁱ+¹ represents a hydrogen atom, an alkyl group possessing 1 to 10 carbon atoms, an aryl group having 5 to 10 carbon atoms, or the silyl alkyl group defined above with formula (II^{o} with

$$i = i + 1 ;$$

i is an integer from 1 to 10 which represents the generation of said silyl alkyl group, and
aⁱ is an integer from 0 to 3;
   where said organic group that can be polymerised by radicals contained in the component (A) is chosen from:
organic groups containing a methacrylic group or a acrylic group and which are represented by the formulas:

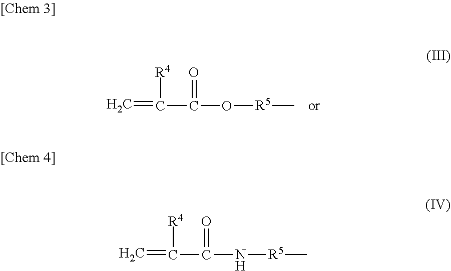

in which:
R⁴ represents a hydrogen atom or an alkyl group with 1 to 10 carbon atoms,
R⁵ represents an alkylene group with 1 to 10 carbon atoms; and
organic groups containing a styryl group of formula:

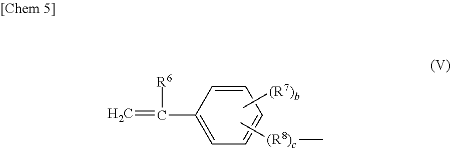

in which:
R⁶ represents a hydrogen atom or an alkyl group with 1 to 10 carbon atoms,
R⁷ represents an alkyl group with 1 to 10 carbon atoms,
R⁸ represents an alkylene group with 1 to 10 carbon atoms,
b is an integer from 0 to 4, and
c has a value of 0 or 1, such that if c as a value of 0, —(R⁸)_{c}— represents a bond.

The vinyl monomer which is the component (A) in the vinyl polymer is a vinyl monomer which contains a vinyl group that can be polymerised by radicals.

There is no particular limitation concerning such a monomer.

The following are examples of this vinyl type monomer: methyl methacrylate, ethyl methacrylate, the n-propyl methacrylate, isopropyl methacrylate, or an analogous lower alkyl methacrylate; glycidyl methacrylate; butyl methacrylate, butyl acrylate, n-butyl methacrylate, isobutyl methacrylate, tert-butyl acrylate, tert-butyl methacrylate, n-hexyl methacrylate, cyclohexyl methacrylate, 2-ethylhexyl acrylate, 2-ethylhexyl methacrylate, octyl methacrylate, lauryl methacrylate, stearyl acrylate, stearyl methacrylate, or an analogous higher methacrylate; vinyl acetate, vinyl propionate, or an analogous lower fatty acid vinyl ester; vinyl caproate, vinyl 2-ethylhexoate, vinyl laurate, vinyl stearate, or an analogous higher fatty acid ester; styrene, vinyltoluene, benzyl methacrylate, phenoxyethyl methacrylate, vinylpyrrolidone, or analogous aromatic vinyl monomers; methacrylamide, N-methylolmethacrylamide, N-methoxymethylmethacrylamide, isobutoxymethoxy-methacrylamide, N,N-dimethyl-methacrylamide, or analogous vinyl monomers which contain amide groups; hydroxyethyl methacrylate, hydroxypropylic alcohol methacrylate, or analogous vinyl monomers which contain hydroxyl groups; acrylic acid, methacrylic acid, itaconic acid, crotonic acid, fumaric acid, maleic acid, or analogous vinyl monomers which contain a carboxylic acid group; tetrahydrofurfuryl methacrylate, butoxyethyl methacrylate, ethoxydiethyleneglycol methacrylate, polyethyleneglycolmethacrylate, polypropyleneglycolmonomethacrylate, hydroxybutyl vinyl ether, cetyl vinyl ether, 2-ethylhexyl vinyl ether, or an analogous vinyl monomer with ether bonds; methacryloxypropyltrimethoxysilane, polydimethylsiloxane having a methacrylic group on one of its molecular ends, polydimethylsiloxane having a styryl group on one of its molecular ends, or an analogous silicone compound possessing unsaturated groups; vinyl chloride butadiene; vinyl chloride; vinylidene chloride; methacrylonitrile; dibutylfumarate; anhydrous maleic acid; anhydrous succinic acid; glycidyl methacryl ether; an organic amine salt, an ammonium salt, and an alkali metal salt of methacrylic acid, itaconic acid, crotonic acid, maleic acid, or fumaric acid; an unsaturated monomer that can be polymerised by radicals possessing a sulfonic acid group such as a styrene sulfonic acid group; a quaternary ammonium salt derived from methacrylic acid such as 2-hydroxy-3-methacryloxypropyltrimethylammonium chloride; and a methacrylic acid ester of an alcohol possessing a tertiary amine group such as a diethylamine methacrylic acid ester.

Multifunctional vinyl monomers can also be used.

The following are examples of such compounds: trimethylolpropane trimethacrylate, pentaerythritol trimethacrylate, ethyleneglycol dimethacrylate, tetraethyleneglycol methacrylate, polyethyleneglycol dimethacrylate, 1,4-butanediol dimethacrylate, 1,6-hexanediol dimethacrylate, neopentylglycol dimethacrylate, trimethylolpropanetrioxyethyl methacrylate, tris-(2-hydroxyethyl) isocyanurate dimethacrylate, tris-(2-hydroxyethyl) isocyanurate trimethacrylate, polydimethylsiloxane capped with styryl groups possessing divinylbenzene on the two ends, or analogous silicone compounds possessing unsaturated groups.

In order to facilitate the preparation of mixtures of raw materials of cosmetic products, the number-average molecular weight of the vinyl polymer which contains a carbosiloxane dendrimer can be chosen in the range between 3000 g/mol and 2,000,000 g/mol, preferably between 5000 g/mol and 800,000 g/mol. It can be a liquid, a gum, a paste, a solid, a powder or in any other form.

The preferred forms are solutions formed by dilution in solvent such as silicone oil or an organic oil, a dispersion, or a powder.

A vinyl polymer contained in the dispersion or solution can have a concentration in a range between 0.1 and 95 wt %, preferably between 5 and 70 wt %. However, in order to facilitate handling and mixture preparation, the range should preferably be between 10 and 60 wt %.

According to a preferred embodiment, a vinyl polymer suitable for the invention can be one of the polymers described in the examples of application EP 0 963 751.

According to a preferred embodiment, a vinyl polymer grafted with a carbosiloxane dendrimer can result from the polymerisation of:
(A) 0.1 to 99 parts by weight of one or more acrylate or methacrylate monomers and
(B) 100 to 0.1 parts by weight of an acrylate or methacrylate monomer of a tri[tri(trimethylsiloxy) silylethyl dimethylsiloxy] silylpropyl carbosiloxane dendrimer.

According to a particular embodiment of the invention, a vinyl polymer having at least one unit derived from carbosiloxane dendrimer comprises a unit derived from tri[tri (trimethylsiloxy) silylethyl dimethylsiloxy] silylpropyl carbosiloxane dendrimer responding to one of formulas

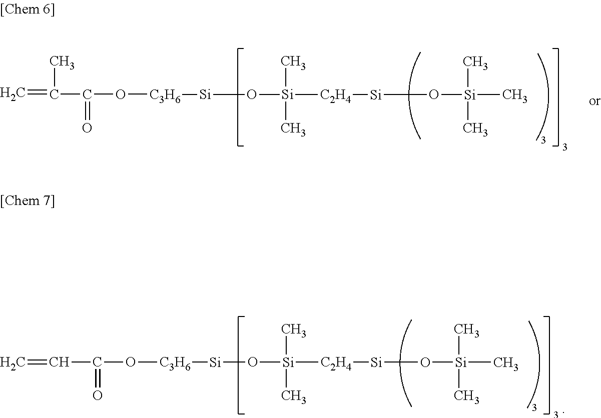

According to a preferred embodiment, a vinyl polymer having at least one unit derived from carbosiloxane dendrimer used in the invention comprises at least one butyl acrylate monomer.

According to a preferred embodiment, a vinyl polymer grafted in the context of the present invention can be transported in an oil or a mixture of one or more, preferably volatile, oils, chosen in particular from silicone oils and hydrocarbon oils, and the mixtures thereof.

According to another particular embodiment, a silicone oil suitable for the invention can be dimethicone.

According to another particular embodiment, a hydrocarbon oil suitable for the invention can be isododecane.

The vinyl polymer grafted with at least one unit derived from carbosiloxane dendrimer that is usable in a composition of the invention is preferably an acrylate/polytrimethylsiloxymethacrylate copolymer with INCI name: ACRYLATES/POLYTRIMETHYL SILOXYMETHACRYLATE COPOLYMER, in particular that marketed in isododecane under the name DOWSIL™ FA 4004 ID Silicone Acrylate by Dow Chemical (40% polymer in 60% isododecane) or that marketed in dimethicone under the name DOWSIL™ FA 4003 ID Silicone Acrylate by Dow Chemical (40% polymer in 60% 2-cst dimethicone).

According to a particular embodiment of the invention, the composition comprises a vinyl polymer having at least one unit derived from carbosiloxane dendrimer with a content of active material (dry matter) ranging from 0.5 to 10 wt %, preferably between 1 and 5 wt %, relative to the weight of the composition.

Silicone Acrylate Polymer (without Dendrimer Unit)

The composition of the invention also comprises a second silicone acrylate polymer b), distinct from compound a) in that it does not comprise a dendrimer unit.

This silicone acrylate polymer is a copolymer comprising methacrylic groups and polydimethylsiloxane groups.

According to the present invention, the term “copolymer comprising methacrylic groups and polydimethylsiloxane groups” shall mean a copolymer obtained from (a) one or more (meth)acrylic acid or (meth)acrylic ester monomers, and (b) one or more polydimethylsiloxane (PDMS) chains.

According to a particular embodiment, the monomer (a) is chosen, for example, from acrylic acid, methacrylic acid, the (acrylate, methacrylate) esters thereof and the mixtures of these monomers. According to a preferred embodiment, the monomers in the form of esters are chosen from linear or branched, preferably C1-C24 or else C1-C22, alkyl acrylates and methacrylates, the alkyl radical being preferably chosen from the methyl, ethyl, stearyl, butyl and ethyl-2-hexyl radicals, and the mixtures thereof.

According to a particular embodiment of the invention, the silicone acrylate copolymer comprises at least one group chosen from acrylic acid, methacrylic acid, methyl, ethyl, stearyl, butyl, ethyl-2-hexyl acrylates or methacrylates, and the mixtures thereof. The term “polydimethylsiloxanes” (PDMS) shall mean any organosilicon polymer or oligomer with linear structure, of variable molecular weight, obtained by polymerisation and/or polycondensation of functionalised silanes, and essentially constituted by a repetition of main units in which the silicon atoms are connected to one another by oxygen atoms (siloxane bond Si—O—Si4), comprising trimethyl radicals directly connected via a carbon atom on said silicon atoms.

The PDMS chains that can be used to obtain the copolymer used according to the invention, comprise at least one polymerisable radical group, preferably located on at least one of the ends of the chain, in other words the PDMS can have, for example, a polymerisable radical group on the two ends of the chain or have a polymerisable radical group on one end of the chain and a trimethylsilyl terminal group on the other end of the chain. The polymerisable radical group can be, in particular, an acrylic or methacrylic group. The copolymers used in the composition of the invention are generally obtained by the usual methods for polymerisation and grafting, for example by radical polymerisation (A) of a PDMS comprising at least one polymerisable radical group (for example, on one or both ends of the chain) and (B) at least one carboxylic monomer, as described for example in documents U.S. Pat. Nos. 5,061,481 and 5,219,560. The copolymers obtained generally have molecular weights ranging from approximately 3000 to 200,000 and preferably approximately 5000 to 100,000. The copolymer used in the composition of the invention can be present per se or in dispersed form in a solvent such as the lower alcohols comprising 2 to 8 carbon atoms, such as isopropylic alcohol, or oils such as volatile silicone oils (e.g. methyl trimethicone) or hydrocarbon oils (e.g. isododecanes). Examples of copolymers include acrylic acid and stearyl acrylate with polydimethylsiloxane grafts, copolymers of stearyl methacrylate with polydimethylsiloxane grafts, copolymers of acrylic acid stearyl methacrylate with polydimethylsiloxane grafts, copolymers of methyl methacrylate, butyl methacrylate, ethyl-2-hexyl acrylate and stearyl methacrylate with polydimethylsiloxane grafts, such as the copolymers marketed by SHIN-ETSU under the name KP-561@ (INCI name: ACRYLATES/STEARYL ACRYLATE/DIMETHICONE METHACRYLATE COPOLYMER), or KP-541® (INCI name: ACRYLATES/DIMETHICONE AND ISOPROPYL ALCOHOL).

According to a particular preferred embodiment, copolymers of polydimethylsiloxane and one or more monomers chosen from acrylic acid, methacrylic acid and the esters are thereof used, such as the products marketed by Shin Etsu under the name KP-550® and with INCI name ACRYLATES/DIMETHICONE COPOLYMER AND ISODODECANE; or KP-549® and KP-579® with INCI name ACRYLATES/DIMETHICONE COPOLYMER AND METHYL TRIMETHICONE.

According to a particular preferred embodiment, the composition of the invention comprises at least one acrylate/dimethicone copolymer in methyl trimethicone, such as KP-549® from Shin-Etsu.

According to an embodiment of the invention, the composition comprises said silicone acrylate polymer (without dendrimer unit) at a content ranging from 0.5 to 7 wt % for the active material (dry matter), in particular 0.8% to 5%, preferably 1% to 3 wt % relative to the total weight of the composition.

According to a particular embodiment of the invention, the weight ratio of dry matter between the silicone acrylate polymer with dendrimer unit and the polymer acrylate without dendrimer unit is greater than or equal to 1. The dry matter weight ratio is preferably between 1 and 3, in particular between 1.5 and 2.

C8-C22 alkyl Dimethicone

The composition of the invention further comprises at least one C8-C22, in particular C12-C20 or even C16-C18 alkyl dimethicone.

According to a particular preferred embodiment, the composition of the invention comprises a cetyl dimethicone (C16).

According to a particular embodiment, the commercial reference ABIL® Wax9840 (INCI name: Cetyl dimethicone) from EVONIK will be used.

The content of alkyl dimethicone, preferably of cetyl dimethicone, in the composition of the invention will generally be 0.05% to 5 wt %, in particular 0.05% to 3 wt %, in particular 0.1% to 2%, preferably 0.5% to 1 wt % relative to the total weight of said composition.

### Aqueous Phase

The aqueous phase of the composition according to the invention generally represents 1 to 50 wt %, preferably 1 to 40 wt %, in particular 5% to 35 wt %, relative to the total weight of said composition.

The aqueous phase comprises water and optionally a water-soluble solvent.

In the context of the invention, “water-soluble solvent” means a compound that is liquid at room temperature and miscible with water (miscibility in water greater than 50 wt % at 25° C. and atmospheric pressure). These include, in particular:
monoalcohols that are less than C₁-C₅ such as ethanol, isopropanol and the mixtures thereof, preferably ethanol;
C₂-C₈ glycols such as ethylene glycol, propylene glycol, 1,3-butylene glycol, dipropylene glycol, and the mixtures thereof;
C₂-C₃₂ polyols such as glycerol, polyglycerols, polyethylene glycols, and the mixtures thereof,
and the mixtures thereof.

Hence, a cosmetic composition according to the invention further comprises at least one water-soluble solvent chosen among the lower C₁-C₅ monoalcohols, C₂-C₈ glycols, C₂-C₃₂ polyols and the mixtures thereof, preferably in a total amount ranging from 8 to 25 wt %, in particular 10 to 20 wt % relative to the total weight of said composition.

According to a particular and preferred embodiment, the composition of the invention will comprise at least ethanol, preferably in an amount ranging from 5 to 20 wt % relative to the total weight of the composition, conferring a fresh effect.

According to a particular preferred embodiment, the composition of the invention will comprise at least glycols as filming agents, in a total content of 1 to 10 wt %, in particular 2 to 5 wt % relative to the total weight of the composition. According to a particular embodiment, the glycol is butylene glycol.

### Colouring Materials

A composition conforming to the present comprises one or more colouring materials which may be chosen from water-soluble or insoluble, liposoluble or insoluble, organic or inorganic colouring materials, materials with optical effects and the mixtures thereof. Within the meaning of the present invention, the term “colouring material” shall mean a compound that is able to produce a coloured optical effect when it is formulated in sufficient quantity in an appropriate cosmetic medium.

According to a particular embodiment, the colouring material or materials are chosen, in particular, among mineral and/or organic pigments, composite pigments (based on mineral and/or organic materials), colouring agents, nacre or nacre pigments, and the mixtures thereof.

The colouring materials are preferably pigments.

“Pigments” shall be understood to be white or coloured, inorganic (mineral) or organic particles, that are insoluble in the organic liquid phase in which they are dispersed, intended to colour, and/or to opacify the composition and/or deposition produced with the composition.

The mineral pigments can include, by way of example, titanium dioxide (rutile or anatase), optionally surface-treated; black, yellow, red and brown iron oxides; manganese violet; ultramarine blue, chromium oxide, hydrated chromium oxide and ferric blue.

The organic pigments can include, for example, pigments D & C red no. 19; D & C red no. 9; D & C Red no. 22; D & C Red no. 21; D & C Red no. 28; D & C Yellow no. 6; D & C orange no. 4; D & C orange no. 5; D & C Red no. 27; D & C red no. 13; D & C Red no. 7; D & C Red no. 6; D & C Yellow no. 5; D & C Red no. 36; D & C Red no. 33; D & C orange no. 10; D & C yellow no. 6; D & C Red no. 30; D &C red no. 3; D &C Blue 1; carbon black and cochineal carmine based lacquers.

The water-soluble colouring agents can include Yellow 5, Yellow 6, Blue 1, Green 5, Green 3, Green 6, Orange 4, Red 4, Red 21, Red 22, Red 27, Red 28, Red 33, Red 40, cochineal carmine (Cl 15850, Cl 75470).

The liposoluble colouring agents are, for example, Sudan red, D&C Red 17, D&C Green 6, beta-carotene, soybean oil, Sudan brown, D&C Yellow 11, D&C Violet 2, D&C orange 5, quinoline yellow and annatto.

The nacres or nacre pigments can be chosen, in particular, from white nacre pigments, such as mica covered with titanium oxide, bismuth oxychloride; and coloured nacre pigments, such as titanium mica with iron oxides, the titanium mica with ferric blue or chromium oxide, titanium mica with an organic pigment of the type mentioned above, as well as bismuth oxychloride-based pigments. Ranges of nacres include those with commercial references Reflecks®, Ronastar®, Timiron® and Syncristal®.

The colouring materials according to the invention and, in particular, the pigments are surface-treated by at least one hydrophobic or lipophilic treatment agent for improved dispersion in the oily phase.

The treatment of the colouring materials generally designates the total or partial surface treatment of the pigment by a surface agent, absorbed or grafted on said pigment, according to techniques known to a person skilled in the art.

The hydrophobic treatment agent is chosen, in particular, from the group consisting of silicone surface agents; fluorinated surface agents; fluorosilicone surface agents; metal soaps, N-acyl amino acids or their salts; lecithin and the derivatives thereof; isopropyl trisostearyl titanate; diisostearyl sebacate; natural plant or animal waxes, synthetic polar waxes; fatty esters; phospholipids, and the mixtures thereof.

According to a particular embodiment, the hydrophobic treatment agent is chosen, in particular, from the group consisting of the silicone surface agents and N-acyl amino acids or the salts thereof.

According to another particular embodiment, the pigments are totally or partially surface-treated with a silicone compound. The silicone surface agents can be chosen from organopolysiloxanes, silane derivatives, silicone-acrylate copolymers, silicone resins, and the mixtures thereof.

By way of example, commercial treatments of pigments with a silicone compound include:
a triethoxycaprylylsilane treatment, such as the AS surface treatment marketed by LCW;
an octyltriethylsilane treatment, such as the OTS surface treatment marketed by Daito;
a methicone treatment, such as the SI surface treatment marketed by LCW;
a dimethicone treatment, such as the Covasil 3.05 surface treatment marketed by LCW or the SA surface treatment from Miyoshi;
a dimethicone/trimethylsiloxysilicate treatment, such as the Covasil 4.05 surface treatment marketed by LCW; and
an acrylate/dimethicone copolymer treatment, such as the ASC surface treatment marketed by Daito.

Preferably, for a surface treatment with a silicone compound, a silane derivative is used such as triethoxycaprylylsilane (AS) or octyltriethylsilane (OTS) treatment, or a organopolysiloxane such as the dimethicone (SI) treatment.

According to a particular preferred embodiment, the colouring materials, and in particular the pigments, are surface-treated by N-acyl amino acids or the salts thereof which can comprise an acyl group having 8 to 22 carbon atoms, such as a 2-ethyl hexanoyl, caproyl, lauroyl, myristoyl, palmitoyl, stearoyl or cocoyl group. The amino acid can be, for example, lysine, glutamic acid or alanine. The salts of these compounds can be aluminium, magnesium, calcium, zirconium, zinc, sodium, or potassium salts.

According to a particularly preferred embodiment, a derivative of N-acyl amino acid can be, in particular, a derivative of glutamic acid and/or one of the salts thereof, and more particularly a stearoyl glutamate, such as aluminium stearoyl glutamate, such as the NAI treatment from Miyoshi with INCI name: disodium stearoyl glutamate (and) aluminium hydroxide, or the treatment from Miyoshi with INCI name: sodium myristoyl glutamate (and) aluminium hydroxide.

According to a particular embodiment, the pigments used are mineral pigments, in particular particles of iron oxides and/or titanium oxide, surface-treated by a treatment agent chosen from the group consisting of disodium stearoyl glutamate, dimethicone, triethoxy caprylylsilane, octyltriethylsilane and the mixtures thereof.

In particular, the one or more colouring materials are present in the composition at a content ranging from 2% to 30 wt %, preferably 4% to 15 wt % relative to the total weight of the composition.

According to a particular embodiment, the composition of the invention comprises pigments, in particular mineral pigments, at a content ranging from 2 to 30%, in particular 4 to 25%, especially 10 to 20 wt % relative to the total weight of the composition.

### Fillers

The composition of the invention can further comprise at least one filler.

Within the meaning of the invention, “fillers” shall mean colourless or white, mineral or organic, natural or synthetic particles, which are insoluble and dispersed in the medium of the composition. The fillers according to the invention may or may not be surface-coated, and, in particular, they can be surface-treated by silicones, amino acids, fluoride derivatives or any other substance promoting the dispersion and compatibility of the filler in the composition.

The fillers are chosen, in particular, from silicas, micas, of natural or synthetic origin, kaolin; calcium carbonate, magnesium carbonate and bicarbonate; zinc, magnesium or lithium stearate, zinc laurate, magnesium myristate, glass and ceramic beads; synthetic polymer powders such as polyethylene, polyesters, polyamides (for example nylon); polyacrylic or polymethacrylic acid powders, silicone resin powders; organic material powders of natural origin such as starches from corn, wheat and rice, cross-linked or not, and the mixtures thereof.

The composition according to the invention will advantageously comprise at least one mineral filler, in particular a silica.

A composition according to the invention can comprise 0.1% to 15 wt %, preferably 2% to 10 wt % of filler(s), relative to the total weight of said composition.

According to a particular embodiment, the composition of the invention comprises:
a) an acrylate/polytrimethylsiloxymethacrylate copolymer, preferably at a content ranging from 0.5 to 10 wt %, preferably between 1 and 5 wt % of dry matter,
b) an acrylate/dimethicone copolymer, preferably at a content ranging from 0.5 to 7%, in particular 0.8 to 5%, preferably 1 to 3 wt % of dry matter,
c) volatile oils, preferably at a total content of at least 10%, or even at least 20 wt %,
d) a cetyl dimethicone, preferably at a content ranging from 0.05% to 5 wt %, in particular 0.05% to 3 wt %, in particular 0.1% to 2%, preferably 0.5% to 1 wt %, and
e) pigments surface-treated by a hydrophobic agent, in particular N-acyl amino acids or the salts thereof, preferably at a content ranging from 2 to 30%, in particular 4 to 25%, especially 10 to 20 wt %
relative to the total weight of said composition.

According to a particularly preferred embodiment, the composition of the invention comprises:
a) an acrylate/polytrimethylsiloxymethacrylate copolymer at a content ranging from 1 to 5 wt % of dry matter,
b) an acrylate/dimethicone copolymer, at a content ranging from 1 to 3 wt % of dry matter,
c) volatile oils at a total content of at least 10%, or even at least 20 wt %,
d) a cetyl dimethicone at a content ranging from 0.5% to 1 wt %, and
e) pigments surface-treated by a hydrophobic agent, in particular N-acyl amino acids or the salts thereof, at a content ranging from 10 to 20 wt % relative to the total weight of said composition.

### Galenic

The composition is preferably intended to be applied on the skin, in particular the skin of the face and/or of the body, and in particular of the face and/or of the neck, and is present in the form of a water-in-oil or water-in-silicone emulsion. It is preferably a water-in-silicone emulsion.

According to a particular preferred embodiment, the composition of the invention is in the form of a foundation, a colour corrector, a concealer, a blush or a colour base, preferably a foundation.

The composition of the invention may also include any additive usually used in cosmetics, such as antioxidants, surfactants, gelling agents, preservatives, perfumes, cosmetic active ingredients, such as, for example, emollients, moisturisers, vitamins, anti-ageing agents, lightening agents, and the mixtures thereof.

### Cosmetic Method

The invention also relates to a cosmetic method for make-up of keratinous materials, in particular the skin, preferably the skin of the face and/or the neck, comprising applying, on said keratinous material, at least one cosmetic composition such as previously defined in the invention.

The invention will be illustrated in the following non-limiting examples. Unless indicated otherwise, the % are expressed in wt % (of raw material) relative to the total weight of said composition.

### EXAMPLE: Selection of the C8-C22 Alkyl Dimethicone for its Effect on the Fidelity and Stability of Colour Over Time

### Preparation of the Composition

The water-in-silicone emulsion is prepared with the following composition:

TABLE 1

| Phase | Ingredients by INCI name | Percentage (%) |
|---|---|---|
| A1 | PEG-9 POLYDIMETHYLSILOXYETHYL DIMETHICONE | 4% |
| | METHYLTRIMETHICONE | 6.46% |
| | METHYL TRIMETHICONE (AND) ACRYLATES/DIMETHICONE | 4% (of which 1.6% |
| | COPOLYMER (KP-549 from Shin Etsu) | dry extract) |
| | ISODODECANE (AND) | 7% (of which 2.8% |
| | ACRYLATES/POLYTRIMETHYLSILOXYMETHACRYLATE | dry extract) |
| | COPOLYMER (DOWSIL ™ FA 4004 ID Silicone Acrylate | |
| | from Dow Chemical) | |
| | ISODODECANE (AND) DISTEARDIMONIUM HECTORITE (AND) | 8% |
| | PROPYLENE CARBONATE (Bentone Gel ® ISD V from | |
| | Elementis) | |
| | ETHYLHEXYL SALICYLATE | 3.5% |
| A2 | TITANIUM DIOXIDE (AND) DISODIUM STEAROYL GLUTAMATE | 8% |
| | (AND) ALUMINIUM HYDROXIDE from Miyoshi | |
| | IRON OXIDES (Red, Yellow and Black) (AND) DISODIUM | 4% |
| | STEAROYL GLUTAMATE (AND) ALUMINIUM HYDROXIDE from | |
| | Miyoshi | |
| | TITANIUM DIOXIDE, TITANIUM DIOXIDE (AND) STEARIC ACID | 4% |
| | (AND) ALUMINA | |
| | Dispersant | 0.5% |
| | METHYL TRIMETHICONE (volatile oil) | 7.43% |
| A3 | SYNTHETIC FLUORPHLOGOPITE | 3% |
| | SILICA | 3% |
| B1 | WATER | q.s.f. 100 |
| | GLYCOLS | 4 |
| | CHLORPHENESIN | 0.2 |
| | Triethanolamine | 0.05 |
| B2 | VP/VA COPOLYMER (PVP-VA S-630 from Ashland) | 0.2 |
| B3 | ETHANOL | 10 |

The composition is obtained according to the following preparation method:
The ingredients of A1 are mixed, then the previously ground ingredients of phase A2 are added; then the ingredients of phase A3 are added;
The ingredients of phases B1, B2 and B3 are mixed;
Phase B is then incorporated in phase A while stirring until a homogeneous emulsion is obtained.

### Evaluation of the Effect of Dispersants on the Fidelity and Stability of the Colour

The following two dispersants were tested: Cetyl dimethicone (ABIL® Wax9840) and respectively lauryl PEG-10 tris(trimethylsiloxy) silylethyl dimethicone (Dowsil ES5300), at 0.5 wt % relative to the total weight of the composition, and the evaluation of the two formulas was compared with a control formula (without dispersant, transfer of 0.5% to methyl trimethicone).

Formula 1 (invention): with 0.5% cetyl dimethicone (ABIL® Wax9840)

Formula 2 (control): without cetyl dimethicone

Formula 3 (comparative): with 0.5% lauryl PEG-10 tris(trimethylsiloxy) silylethyl dimethicone

The three formulas have thus been evaluated and compared, two-by-two, on a half-face, by a panel of colourist experts according to the following protocol. Application of the products was carried out by the cosmetic expert with a brush on the half-face, and the following observations were made:
Observation of the bulk colour under the slide (corresponding to the colour at the outlet of the pump)
Observation of the colour after application on the skin of the face (T5-30 min)
Observation of the change in the colour over time (T2 h and T6 h)

The tests are carried out in absolute value on powder-free skin with a moisturising cream as base. The evaluations are made using VISIA-CR, visualisation mode (eyes closed), from the front and left profile for the faces. The photographs were taken at application (T5 minutes, immediately after application), T15 min, T2 h and T6 h.

Two criteria were evaluated by the panel of experts:
the ‘colour placement’ at application, corresponding to the make-up result (colour) obtained after putting on. The definitive result is generally obtained at between 5 and 30 minutes (evaluated here at T5 minutes after application and T15 minutes). A colour is sought after application which is as faithful as possible to the colour at the outlet of the pump (colour in the bulk). Reference is made to the ‘colour fidelity’ at application; and
the ‘rise in colour’ over time corresponding to the change in the colour over the course of the day (evaluated here at T2 h and T6 h). A colour is sought after 6 hours that is as faithful as possible to the colour after application, in other words a low rise in colour over time. Reference is made to “colour fidelity” or “colour stability” over time.

The results are presented in the following table:

TABLE 2

| Formula | | |
|---|---|---|
| number | Formula | Evaluation of the expert panel |
| Formula 1 | Formula with 0.5% cetyl | In bulk under slide: slightly hotter compared |
| (invention) | dimethicone ((ABIL ® | with formulas 2 and 3 |
| | Wax9840)) | At putting on: slightly more yellow vs. |
| | | formula 2 |
| | | After 15 min: good fidelity of the colour, |
| | | the film remains homogeneous, covering and |
| | | luminous |
| | | T2 h and T6 h: the holding of the film is |
| | | satisfactory. A good correction and coverage |
| | | is observed and a low rise in colour. |
| Formula 2 | Control formula (without | In bulk under slide: slightly clearer and more |
| (control) | dispersant) | faded than formula 1 |
| | | At putting on: slightly dull in the grey red vs. |
| | | formula 1 |
| | | After 15 min: slightly duller vs. formula 1 |
| | | T2 h and T6 h: the holding of the film is |
| | | satisfactory, but slight change in the colour |
| | | (duller). |
| Formula 3 | Formula with Lauryl PEG-10 | In bulk under slide: slightly lighter in the |
| (comparative) | tris(trimethylsiloxy)silylethyl | yellow vs. formula 1 |
| | dimethicone (Dowsil ES5300) | At putting on: slightly more yellow vs. |
| | | formula 1 |
| | | After 15 min: slightly more yellow vs. |
| | | formula 1 |
| | | T2 h and T6 h: the holding of the film is |
| | | satisfactory, but slight change in the colour |
| | | (more yellow). |

These results show that cetyl dimethicone (C8-C22 alkyl dimethicone) in the formula 1 according to the invention makes it possible to obtain better performance in terms of fidelity of the colour after application and stability of the colour over the course of the day, compared with formula 2 (control) without dispersant (colour duller after application and over time) or with formula 3 (comparative) with another dispersant (colour more yellow after application and over time).

The use of a cetyl dimethicone in a composition according to the invention therefore enables a better dispersion of pigments that are surface-treated by a hydrophobic agent, for a make-up result (colour) that is faithful and stable over time, while preserving the properties of non-transfer of the composition linked with the combination of 2 silicone acrylate polymers.

1. A cosmetic make-up composition for keratinous materials, in the form of a water-in-oil emulsion, comprising, in a physiologically acceptable medium, at least:
a) a vinyl polymer having at least one unit derived from carbosiloxane dendrimer,
b) a silicone acrylate polymer different from compound a),
c) one or more volatile oils,
d) a C8-C22 alkyl dimethicone, and
e) colouring materials, surface-treated by a hydrophobic agent.

2. The composition according to claim 1, wherein the vinyl polymer having at least one unit derived from carbosiloxane dendrimer, possesses a molecular side-chain containing a carbosiloxane dendrimer structure, and originates from the polymerisation of:
(A) 0 to 99.9 parts by weight of a vinyl monomer; and
(B) 100 to 0.1 parts by weight of a carbosiloxane dendrimer containing an organic group that can be polymerised using radicals, represented by the general formula (I): 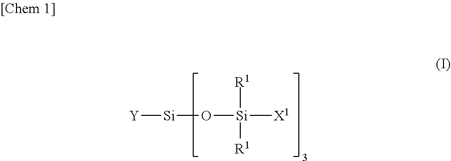
wherein:
Y represents an organic group that can be polarised by radicals,
R1 represents an aryl group with 5 to 10 carbon atoms or an alkyl group possessing 1 to 10 carbon atoms, and
Xi represents a silyl alkyl group which, when i=1, is represented by the formula (II): 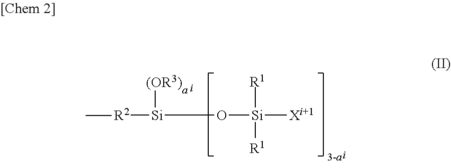
wherein:
R¹ is as in formula (1),
R² represents an alkylene group possessing 2 to 10 carbon atoms,
R³ represents an alkyl group possessing 1 to 10 carbon atoms,
Xⁱ+¹ represents a hydrogen atom, an alkyl group possessing 1 to 10 carbon atoms, an aryl group with 5 to 10 carbon atoms, or the silyl alkyl group defined above with formula (II), with i=i+1;
i is an integer from 1 to 10, which represents the generation of said silyl alkyl group, and
aⁱ is an integer from 0 to 3;
where said organic group that can be polymerised by radicals contained in the component (A) is chosen from:
organic groups containing a methacrylic group or an acrylic group and which are represented by the formulas: 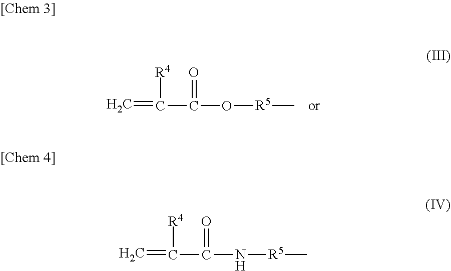
in which:
R⁴ represents a hydrogen atom or an alkyl group with 1 to 10 carbon atoms,
R⁵ represents an alkylene group with 1 to 10 carbon atoms; and
organic groups containing a styryl group of formula: 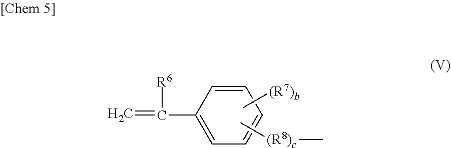
in which:
R⁶ represents a hydrogen atom or an alkyl group with 1 to 10 carbon atoms,
R⁷ represents an alkyl group with 1 to 10 carbon atoms,
R⁸ represents an alkylene group with 1 to 10 carbon atoms,
b is an integer from 0 to 4, and
c has a value of 0 or 1, such that if c is 0, —(R⁸)_{c}— represents a bond.

3. The composition according to claim 2, wherein the vinyl polymer comprises a unit derived from tri[tri(trimethylsiloxy) silylethyl dimethylsiloxy] silylpropyl carbosiloxane dendrimer responding to formulas

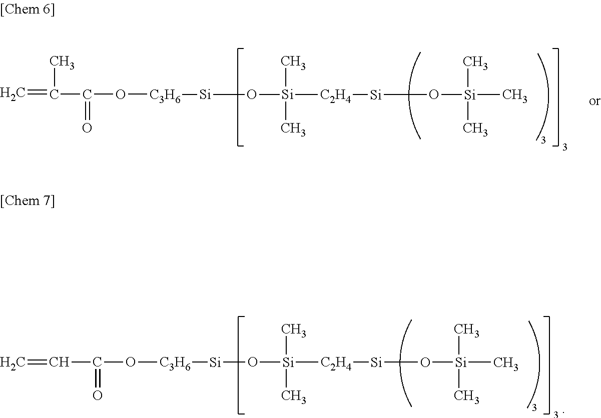

4. The composition according to claim 1, wherein the vinyl polymer having at least one unit derived from carbosiloxane dendrimer corresponds to an acrylate/polytrimethylsiloxymethacrylate copolymer with INCI name: acrylate/polytrimethylsiloxymethacrylate copolymer.

5. The composition according to claim 1, wherein the silicone acrylate polymer b) distinct from compound a) includes methacrylic groups and polydimethylsiloxane groups.

6. The composition according to claim 1, wherein the C8-C22 alkyl dimethicone is a cetyl dimethicone.

7. The composition according to claim 1, wherein the colouring materials are pigments that are surface-treated by a hydrophobic agent chosen from the group consisting of silicone surface agents, N-acyl amino acids and the salts thereof.

8. The composition according to claim 1, which comprises:
a) an acrylate/polytrimethylsiloxymethacrylate copolymer, preferably at a content ranging from 0.5 to 10 wt %, preferably between 1 and 5 wt % of dry matter,
b) an acrylate/dimethicone copolymer, preferably at a content ranging from 0.5 to 7%, in particular 0.8 to 5%, preferably 1 to 3 wt % of dry matter,
c) volatile oils, preferably at a total content of at least 10%, or of at least 20 wt %,
d) a cetyl dimethicone, preferably at a content ranging from 0.05% to 5 wt %, especially 0.05% to 3 wt %, in particular 0.1% to 2%, preferably 0.5% to 1 wt %, and
e) pigments that are surface-treated by a hydrophobic agent, in particular N-acyl amino acids or the salts thereof, preferably at a content ranging from 2 to 30%, in particular 4 to 25%, especially 10 to 20 wt %
with respect to the total weight of said composition.

9. The composition according to claim 1, which further comprises one or more glycols.

10. A method for making-up keratinous materials, comprising the application on said keratinous material, of a cosmetic make-up composition for keratinous materials, in the form of a water-in-oil emulsion, comprising, in a physiologically acceptable medium, at least:
a) a vinyl polymer having at least one unit derived from carbosiloxane dendrimer
b) a silicone acrylate polymer different from compound a),
c) one or more volatile oils
d) a C8-C22 alkyl dimethicone, and
e) colouring materials, surface-treated by a hydrophobic agent.

11. The composition according to claim 5, wherein the silicone acrylate polymer b) distinct from compound a) is chosen from the acrylate/dimethicone copolymers.

12. The composition according to claim 8, wherein:
a) the acrylate/polytrimethylsiloxymethacrylate copolymer is present at a content ranging from 0.5 to 10 wt % of dry matter,
b) the acrylate/dimethicone copolymer is present at a content ranging from 0.5 to 7 wt % of dry matter,
c) the volatile oils are present at a total content of at least 10% wt %,
d) the cetyl dimethicone is present at a content ranging from 0.05% to 5 wt %, and
e) pigments that are surface-treated by a hydrophobic agent, are present at a content ranging from 2 to 30 wt %
with respect to the total weight of said composition.

13. The method for making-up keratinous materials according to claim 10, wherein the composition is applied in the skin.
